(19) United States
(12) Patent Application Publication
(10) Pub. No.: US 2012/0094373 A1
(43) Pub. Date: Apr. 19, 2012

(54) CONTAINER FOR NUCLEIC ACID AMPLIFICATION REACTION

(75) Inventors: Chen SU, Taichung (TW); Ping-Hua TENG, Taichung (TW)

(73) Assignee: GENEREACH BIOTECHNOLOGY CORP., Taichung (TW)

(21) Appl. No.: 13/013,831

(22) Filed: Jan. 26, 2011

(30) Foreign Application Priority Data
Oct. 14, 2010 (TW) 099135105

(51) Int. Cl. C12M 1/40

(52) U.S. Cl. 435/304.1

(57) ABSTRACT

A container for nucleic acid amplification reaction is disclosed. The container includes a capillary and a conductor. The conductor tightly surrounds the capillary for heating the capillary evenly.

### RELATED APPLICATIONS

The application claims priority to Taiwan Application Serial Number 99135105, filed Oct. 14, 2010, which is herein incorporated by reference.

### BACKGROUND

1. Technical Field

The present disclosure relates to nucleic acid amplification reaction. More particularly, the present disclosure relates to a container for nucleic acid amplification reaction.

2. Description of Related Art

The nucleic acid amplification reaction is a scientific technique in molecular biology to amplify a single or a few copies of a particular deoxyribonucleic acid (DNA) sequence by repeating the same procedure with particular polymerases. The common techniques such as polymerase chain reaction (PCR), reverse transcription polymerase chain reaction (RT-PCR), and real-time polymerase chain reaction (real-time PCR) all belong to nucleic acid amplification reaction techniques.

The PCR is used to amplify a particular DNA. The RT-PCR is used to amplify a particular DNA, which is copied by a template, complementary DNA (cDNA), wherein the cDNA is reverse transcribed from a Ribonucleic acid (RNA). The real-time PCR, also called quantitative PCR, is used to amplify and simultaneously quantify a targeted DNA, wherein the methods for quantification are fluorescent probe and dyes. Therefore, the fundamental skill of the nucleic acid amplification reactions is PCR.

Furthermore, some skills presented lately also belong to nucleic acid amplification reactions, such as rolling circle amplification (RCA), loop mediated amplification (LAMP), nucleic acid sequence based amplification (NASBA), and three way junction (TWJ).

About PCR, the Initialization step is used for mixing and heating DNA templates, primers, and a buffer solution to the reaction temperature about 90° C. for disrupting the hydrogen bonds between two single-stranded DNA templates. The second step is used for cooling the reaction temperature to about 50° C. for annealing the primers and the single-stranded DNA template. The final step is used for holding the temperature at about 70° C. for extending the primers. The particular DNA is copied by repeating the above procedure.

The types of the apparatus for the nucleic acid amplification reaction are classified according to the prices. The cheap type includes a container, such as a tube or a capillary, and two heaters. The two heaters are respectively disposed on the two ends of the container. One heater heats the container to about 90° C., and the other heats the container to about 50° C. The solution convection in the container takes place because of the density difference of the solution at the two ends of the container, wherein the density difference is caused by the temperature difference between the two ends. The DNA and the primers is circulated through the container and heated from 90° C. to 50° C. circularly for performing the nucleic acid amplification reaction.

The heater is made of a metal block. The block has a groove for receiving the end of the container, and the shape of the groove is similar to the end of the container. However, the groove does not match with the container. It means that the groove has some protrusions and indentations when the end of the container is received in the groove. Therefore, the edge of the protrusions and the indentations do not touch the container and conduct heat to the container. Thus, the container is not heated evenly. The reaction rate of the nucleic acid amplification reaction will be reduced.

### SUMMARY

According to one embodiment of the present disclosure, a container for nucleic acid amplification reaction includes a capillary and a conductor. The conductor tightly surrounds the capillary for heating the capillary evenly.

### BRIEF DESCRIPTION OF THE DRAWINGS

FIG. 1 is a three-dimensional view of a container according to one embodiment of the present disclosure.

FIG. 2 is an explosive view of the container of FIG. 1.

FIG. 3 is a cross-sectional view viewed along line 3-3 of FIG. 1.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

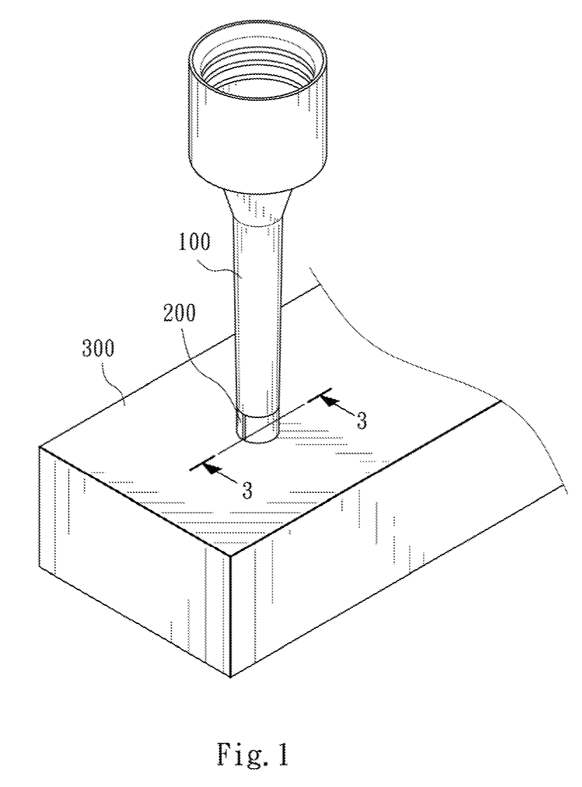

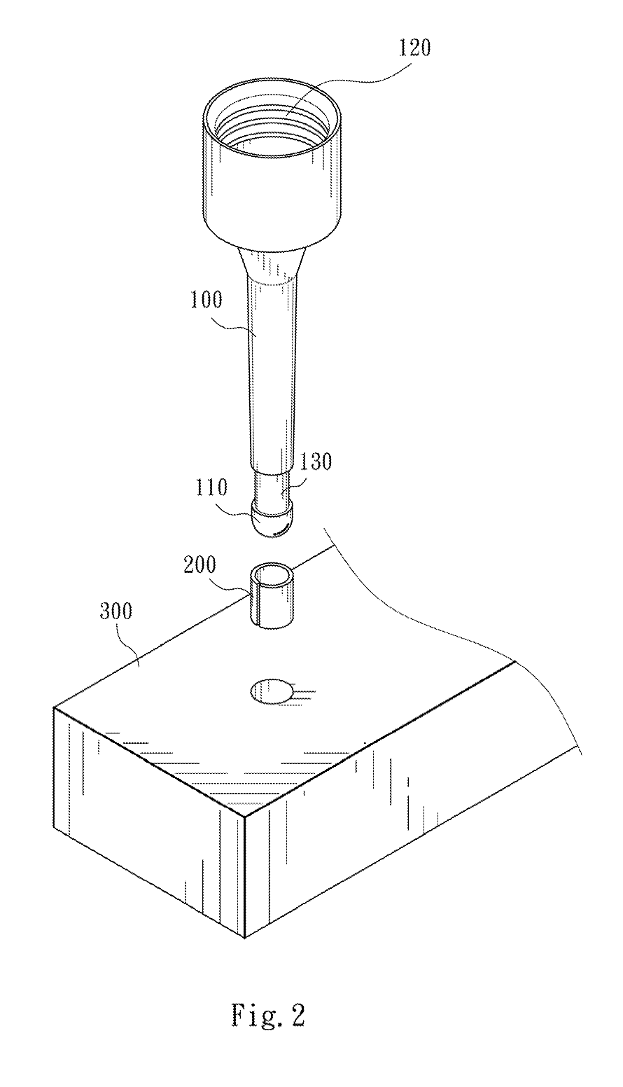

FIG. 1 is a three-dimensional view of a container according to one embodiment of the present disclosure. FIG. 2 is an explosive view of the container of FIG. 1. The container includes a capillary 100 and a conductor 200. The conductor 200 tightly surrounds on the capillary 100.

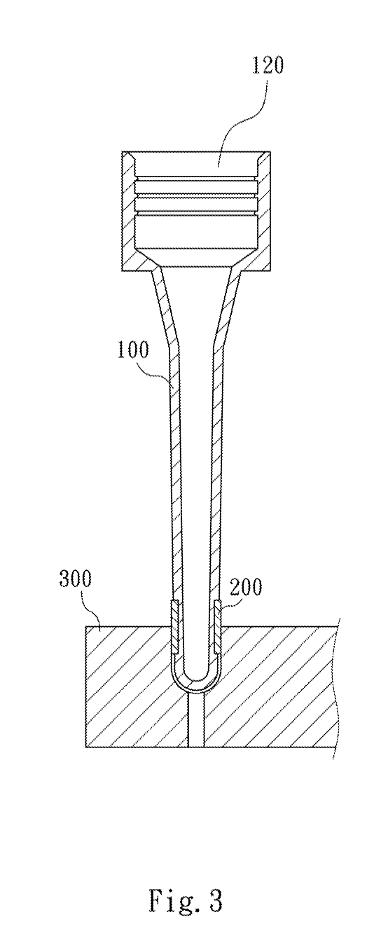

FIG. 3 is a cross-sectional view viewed along line 3-3 of FIG. 1. The conductor 200 tightly surrounds one end 110 of the capillary 100, wherein the end 110 is close, and the other end 120 is open. In use, the conductor 200 absorbs the heat from a heater 300, and the heat is conducted to the capillary 100. The nucleic acid amplification reaction is performed in the capillary 100 when the temperature of the end 110 is about 90° C. controlled by the heater 300 and the temperature of the other end 120 is about 50° C. controlled by the environment. For instance, the temperature of the end 110 is about 90° C., and the room temperature is lower than 50° C. Therefore, the other end 120 dissipates the heat to the environment for controlling the temperature to about 50° C. Furthermore, the room temperature is controlled by a thermostat system (not shown).

The heat is evenly conducted from the conductor 200 to the capillary 100 because of the conductor 200 tightly surrounding the capillary 100. Furthermore, the capillary 100 is separated from the heater 300 and heated by the conductor 200. Therefore, the capillary 100 is heated evenly for increasing the reaction rate of the nucleic acid amplification reaction.

As shown in FIG. 2, the capillary 100 includes an annular groove 130. The annular groove 130 is positioned at the end 110 for receiving and positioning the conductor 200. The conductor 200 is a clip, and the inner diameter of the conductor 200 is less than or equal to the outer diameter of the capillary 100. Thus, the conductor 200 expands and snaps into the annular groove 130 of the capillary 100. In detail, the conductor 200 is a circlip. Furthermore, the conductor 200 also is a sleeve, and the inner diameter of the conductor 200 is equal to the outer diameter of the capillary 100. Thus, the conductor 200 encircles the annular groove 130 of the capillary 100 tightly. In detail, the conductor 200 is an expansion sleeve. Particularly, all of the inner side of the conductor 200 touches the outside of the capillary 100, no matter what the conductor 200 is a clip or a sleeve.

The capillary 100 is made of plastic. In detail, the capillary 100 is made of polycarbonate (PC). The conductor 200 is made of metal. In detail, the conductor 200 is made of iron. The material of the capillary 100 and the conductor 200 not only correspond to the standard of the heat-resistant and the strength, but also reduce the price.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. §112, 6th paragraph. In particular, the use of “step of” in the claims is not intended to invoke the provisions of 35 U.S.C. §112, 6th paragraph.

What is claimed is:

1. A container, comprising:
a capillary; and
a conductor tightly surrounding the capillary for heating the capillary evenly, wherein the conductor absorbs the heat from a heater.

2. The container of claim 1, wherein the conductor is a clip.

3. The container of claim 2, wherein the conductor is a circlip.

4. The container of claim 1, wherein the conductor is a sleeve.

5. The container of claim 4, wherein the conductor is an expansion sleeve.

6. The container of claim 1, wherein the capillary is made of plastic.

7. The container of claim 6, wherein the capillary is made of polycarbonate.

8. The container of claim 1, wherein the conductor is made of metal.

9. The container of claim 8, wherein the conductor is made of iron.

10. The container of claim 1, wherein the capillary comprises an annular groove positioned at one end of the capillary for receiving and positioning the conductor.

11. The container of claim 1, further comprising a thermostat system for controlling the room temperature, wherein the room temperature absorbs the heat of the capillary.
